(19) United States
(12) Patent Application Publication
(10) Pub. No.: US 2016/0151525 A1
(43) Pub. Date: Jun. 2, 2016

(54) SYSTEM AND METHOD FOR TREATMENT OF PERISHABLE GOODS WITH HYDROGEN-RICH WATER

(71) Applicant: PRODEW, INC., Marietta, GA (US)

(72) Inventors: Itamar KLEINBERGER, Marietta, GA (US); Shakeel MERCHANT, Atlanta, GA (US); Faizan ALY, Smyrna, GA (US); Roland BILODEAU, Canton, GA (US); David SCHULZE, Marietta, GA (US)

(73) Assignee: PRODEW, INC., Marietta, GA (US)

(21) Appl. No.: 14/953,946

(22) Filed: Nov. 30, 2015

Related U.S. Application Data
(60) Provisional application No. 62/085,754, filed on Dec. 1, 2014.
(60) Provisional application No. 62/126,894, filed on Mar. 2, 2015.

(51) Int. Cl. A61L 2/22; C02F 1/467

(52) U.S. Cl. CPC A61L 2/22; C02F 1/4676

(57) ABSTRACT

A system for preserving and disinfecting perishables or other items, equipment or objects. In example forms, a humidification and treatment system includes an electrolysis device for converting a supply of water into hydrogenated sterilized water, and the hydrogenated sterilized water is discharged into an environment in the form of a mist and/or a fog to disinfect, clean, preserve and/or hydrate the perishables and/or the environment associated therewith.

### CROSS-REFERENCE TO RELATED APPLICATIONS

This application claims the priority benefit of U.S. Provisional Patent Application Ser. No. 62/085,754 filed Dec. 1, 2014 and U.S. Provisional Patent Application Ser. No. 62/126,894 filed Mar. 2, 2015, the entireties of which are hereby incorporated herein by reference for all purposes.

### TECHNICAL FIELD

The present invention relates generally to water treatment and humidification, and more particularly to systems and methods for delivery of hydrogen rich water by misting or fogging to preserve perishable items and/or for disinfection or sterilization of items and control of pathogens. The invention also relates to systems and methods of preservation of flowers, floral arrangements, produce, cannabis, seafood, meat, and other perishable plant or animal matter using hydrogen rich water.

### BACKGROUND

Water misting and humidification delivery systems are used to maintain freshness of perishable items such as produce, meats and seafood, and flowers in grocery stores and other areas. Misting or fogging systems commonly deliver periodic sprays of water droplets or micro-droplets to the maintained products to maintain hydration, freshness and crispness. It has been found, however, that without proper controls, humidification could potentially also promote the growth of pathogenic organisms (e.g., fungi, mold, mildew, bacteria, yeast, etc.), which may naturally or otherwise be present in, on or around the products to be preserved, which can have detrimental effects on the products or their marketability.

Known methods of controlling pathogens include the delivery of heat for sterilization, the use of high energy/high temperature catalytic conversion systems, the use of steam or microwaves, the use of sprayed or fogged or bubbling systems employing chemicals such as hypochlorite or peracetic acid, the injection of ozone into the air, or the use of medical grade HEPA filters. Many known methods of controlling pathogens can be harmful to people or cause corrosion of equipment. Additional methods such as photo catalytic oxidation systems using titanium dioxide and other photo catalytic substances have shown promise, but they typically require a very slow flow rate to effectively process the air and can be high maintenance. Ozone disinfecting systems that employ a catalyst after the ozone has been generated are typically limited to a very low flow that allows the catalyst to work. Germicidal ultraviolet (UV) light can be used for disinfection if a modest flow rate is maintained and care is taken to prevent skin and eye exposure to the rays, but higher flow rates may allow spore states of pathogenic organisms to remain viable.

Accordingly, it can be seen that needs exist for environmentally friendly and non-toxic systems and methods of disinfecting and controlling pathogens. Such needs are particularly acute in and around perishable items subject to damage by pathogenic organisms. For example, the reduction of losses of perishables to rots and molds is directly linked to their maintenance in areas free from opportunistic bacterium such as Pseudomonas lundensis and molds such as botrytis and penicillium. It is to the provision of systems and methods meeting these and other needs that the present invention is primarily directed.

### SUMMARY

The present invention provides improved systems and methods for disinfecting and controlling pathogens by misting or fogging delivery of hydrogen-rich water. In example embodiments, the present invention provides a humidification system comprising an electrolysis device for converting a supply of water into hydrogenated or hydrogen-rich sterilized water (sometimes referred to as hydrogen water), and a delivery system for discharging the hydrogen sterilized water into an environment in the form of a mist or a fog to control pathogens, to disinfect a treated object or area, and/or to provide hydration for treated items or materials.

In one aspect, the present invention relates to a misting humidification and treatment system including a water supply, a misting tank, an electrolysis device, and a misting track. A control system is optionally provided for control of delivery parameters such as for example flow-rate, delivery timing and duration, hydrogen concentration, and/or droplet size. Preferably, the electrolysis device converts water from the water supply into hydrogenated sterilized water. The misting track preferably includes at least one nozzle for discharging the hydrogenated sterilized water therefrom in droplet or micro-droplet form.

In another aspect, the invention relates to a humidification system including a control box, a humidity solenoid, a hydrogen water delivery source, a compressor, and a fog/mist track. The fog/mist track preferably includes at least one nozzle for discharging the hydrogenated water therefrom in fog or mist form.

In still another aspect, the invention relates to a fogging humidification and treatment system including a control system, a humidity solenoid, an electrolysis device, a compressor, and at least one fogging element. The electrolysis device is preferably provided for converting a supply of water into hydrogen-rich sterilized water. The at least one fogging element includes at least one nozzle for discharging the hydrogenated water therefrom in fog form.

In yet another aspect, the present invention relates to a system for preserving perishables. The system preferably includes a humidification system having an electrolysis device for converting a supply of water into hydrogenated sterilized water. In example forms, the hydrogenated sterilized water is discharged into an environment in the form of a mist and/or a fog to disinfect, clean, preserve and hydrate the perishables and/or the environment associated therewith.

In yet another aspect, the present invention relates to a method for controlling pathogens. The method preferably includes the provision of a quantity of hydrogen-rich water, conversion of the hydrogen-rich water into a mist or fog form, and the delivery of the hydrogen-rich water mist or fog to a treated object or area.

In another aspect, the present invention relates to a system for preserving perishables such as flowers, produce, cannabis, seafood, meat, or other perishable plant or animal matter. The system includes one or more vases or containers for containing flowers, other plants, or other perishable items with at least a portion of the perishable items in contact with a quantity of hydrogen rich water. In example forms, hydrogen water cells are included in each of one or more vases or containers in the system. In alternate embodiments, hydrogen rich water is circulated or otherwise delivered to a plurality of vases or other containers from a shared source of hydrogen rich water, and the system includes the shared source of hydrogen rich water, a plurality of vases or containers, and piping or fluid delivery conduits for distribution of the hydrogen rich water from the source to the vases or containers.

In another aspect, the present invention relates to a method for preserving perishables such as flowers, produce, cannabis, seafood, meat, or other perishable plant or animal matter. The method includes placement of flowers, other plant matter, or other perishable items in one or more containers with at least a portion of the perishable items in contact with a quantity of hydrogen rich water. In various example forms, the hydrogen rich water is produced in, contained in, and/or circulated or distributed to the one or more containers.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a schematic showing a humidification and treatment system delivering hydrogen sterilized water according to an example embodiment of the present invention.

FIG. 2 is a schematic showing a humidification and treatment system delivering hydrogen sterilized water according to another example embodiment of the present invention.

FIG. 3 is a schematic showing a humidification and treatment system delivering hydrogen sterilized water according to another example embodiment of the present invention.

FIG. 4 is a diagram showing the reaction of water within an electrolysis device to form hydrogen-rich sterilized water.

FIG. 5 shows a system and method for preserving flowers, floral arrangements and/or other plant matter using hydrogen-rich water.

### DETAILED DESCRIPTION OF EXAMPLE EMBODIMENTS

The present invention may be understood more readily by reference to the following detailed description of example embodiments of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

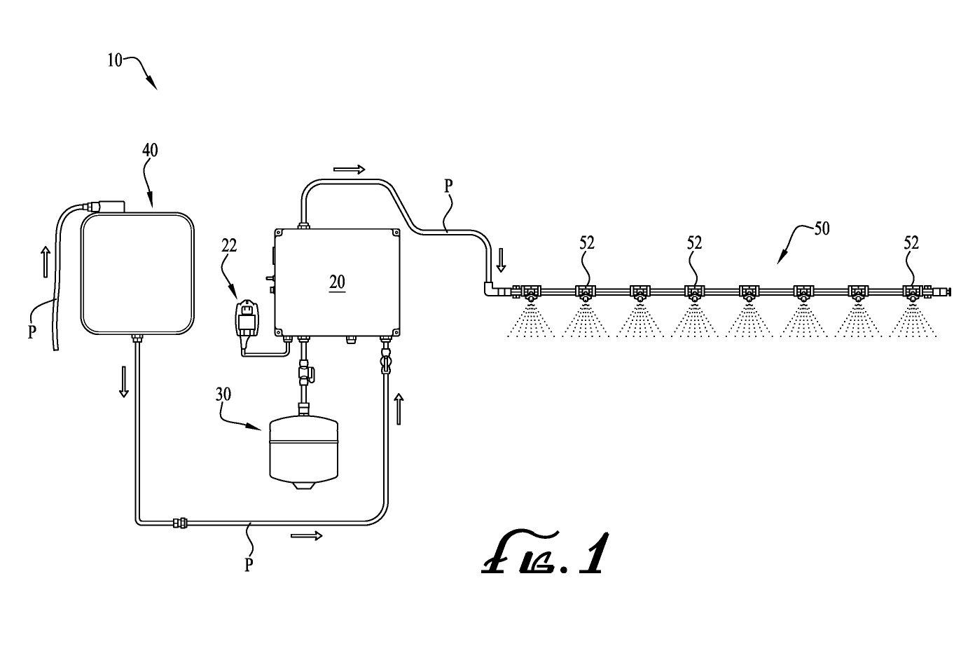

With reference now to the drawing figures, wherein like reference numbers represent corresponding parts throughout the several views, FIG. 1 shows an example embodiment of a humidification and treatment system 10 according to an example embodiment of the present invention. The system 10 generally includes a control subsystem or control box 20, a misting tank 30, an electrolysis device or hydrogen water processing subsystem (hydrogen box) 40, and at least one misting track 50. In the depicted embodiment, the control subsystem 20 preferably includes an electronic control system and valving for controlling the operation of the system and the delivery of hydrogenated water from the hydrogen water subsystem 40, into the misting tank 30, and out of a plurality of misting or fogging nozzles 52 of the misting track 50. The hydrogenated water is preferably delivered in mist or fog form to a treated object or area, for example to disinfect or control pathogens in, on or around the treated object or area. In example embodiments, the treated object or area includes a containment, delivery, display or storage area for perishable items, such as for example, flowers, produce, cannabis (marijuana), seafood, meat, or other perishable plant or animal matter. In example embodiments, the hydrogenated water droplets being discharged from the nozzles 52 are each about 100 microns in size. Optionally, the nozzles 52 may be adjusted to provide hydrogenated water droplets of other desired sizes, for example, between about 50-200 microns.

In example forms, the control subsystem 20 preferably comprises an electrical plug 22 for receiving electricity from a power outlet or other power source. Preferably, the control box 20 comprises a plurality of electronic components including one or more circuit boards, micro controllers, chips, processors, timers, switches, etc., and fluid conduits and valving, to provide for optimum functionality and ease of use of the system 10. One or more user inputs, displays or interfaces are optionally provided for selective control by an operator. Piping P or other fluid delivery conduits are provided between the components to deliver regular water (before moving through the hydrogen box 40) and hydrogenated water (after leaving the hydrogen box 40) to be output from the nozzles 52 of the misting track 50. A variety of couplings, valves, gaskets, washers, fittings, etc. may be provided for coupling the piping P to the elements of the system 10 or for coupling two or more pieces of piping together. In example forms, the piping P can be configured, formed and sized as specified to fit a particular application. Optionally, the system can comprise a plurality of misting tracks 50, for example arranged in parallel or in series, coupled to piping P for delivery of the hydrogen water to two or more treated objects or areas. In example forms, each misting track 50 comprises at least one, and optionally a plurality of nozzles 52 spaced along its length.

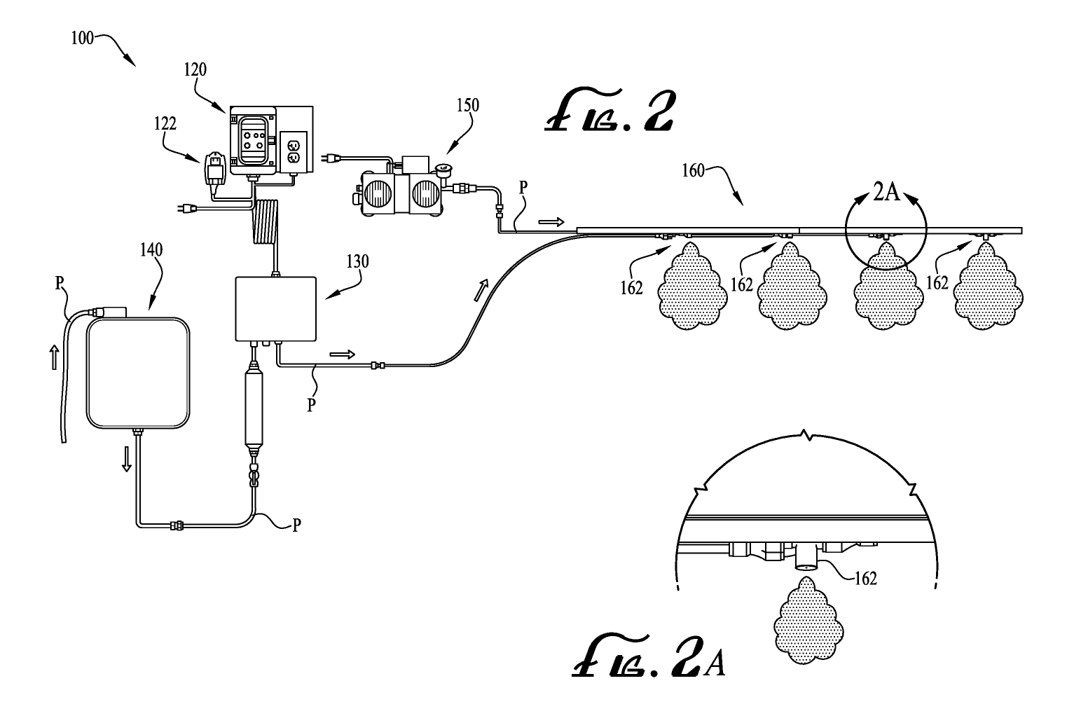

FIG. 2 shows another example embodiment of a humidification and treatment system 100 according to the present invention. As depicted, the humidification system 100 generally comprises a control subsystem or control box 120, a humidity solenoid 130, a hydrogen box or hydrogen water processing subsystem 140, a compressor 150, and at least one fog/mist track 160. Electronic controls in the control subsystem 120 actuate the solenoid 130 to deliver hydrogen sterilized water from the hydrogen subsystem 140, and the compressor 150 delivers the hydrogen water to the fog/mist track 160 under pressure to generate the fog or mist of hydrogen water droplets. In the depicted embodiment, the fog/mist track comprises a plurality of nozzles 162 that generally discharge hydrogenated water in a stream or spray of droplets in the range of about 20-150 microns. According to an example embodiment, the hydrogenated water droplets are about 50 microns in size.

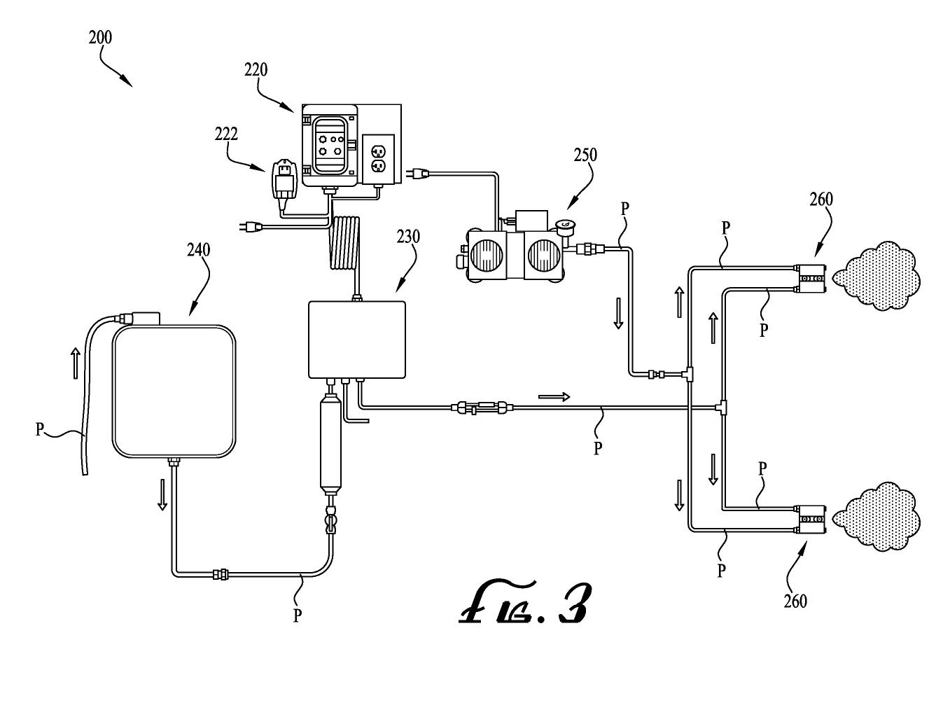

FIG. 3 shows a humidification and treatment system 200 according to another example embodiment of the present invention. In similar fashion to that described above, the humidification system 200 generally comprises a control subsystem or control box 220, a humidity solenoid 230, a hydrogen water processing subsystem or hydrogen box 240, a compressor 250, and a plurality of fogging elements 260. Preferably, the fogging elements 260 each comprise one or more nozzles affixed thereon for producing a fog or very fine or subtle mist of hydrogenated water droplets between about 2-20 microns in size. According to one example embodiment, the fogging elements 260 produce hydrogenated water droplets between about 6-8 microns. Optionally, the hydrogenated water droplets can be varied in size (e.g., larger or smaller) as desired.

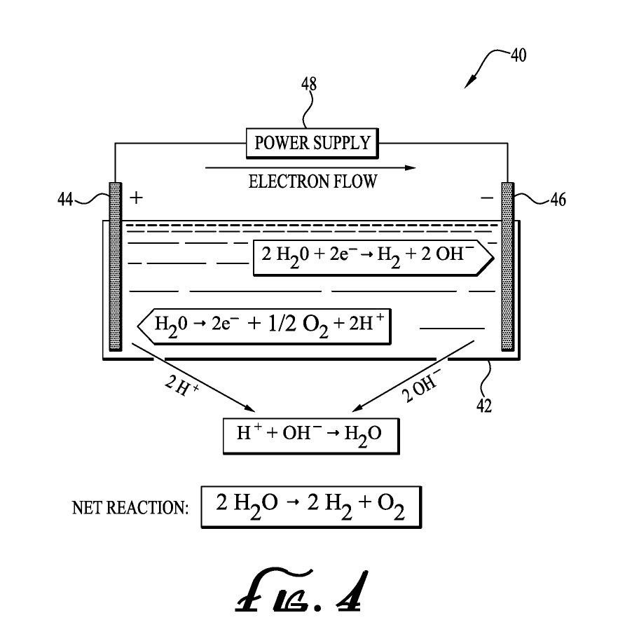

In example embodiments, the hydrogen subsystem 40, 140, 240 incorporates an electrolysis system in connection with the input of water to produce hydrogen-rich water having a higher content of dissolved or solubilized available molecular hydrogen gas than untreated water (sometimes referred to as hydrogen water, hydrogenated water, hydrogen rich water, or hydrogen sterilized water). For example, with reference to FIG. 4, a hydrogen subsystem according to example embodiments preferably comprises a tank or other container 42 for holding water, a positive electrode or charged plate (anode) 44, a negative electrode or charged plate (cathode) 46, and a power source 48 such as a direct current (DC) battery, generator or other power source. Optionally, an electrolysis catalyst or electrolyte (such as a salt, an acid or a base) is also provided. The electrodes may comprise, for example, platinum, stainless steel, iridium, or other inert metal plates. Hydrogen is generated at the cathode, and the resultant hydrogen rich water surrounding the cathode is collected and distributed by the hydrogen subsystem.

Generally, the generation of hydrogen in water is a result of the oxidizing potential of water molecules dissociated (“ionized”) into hydrogen ions and hydroxide ions:

H₂O→H⁺+OH⁻

The resulting H and OH have the ability to kill microorganisms, and oxidize organic particles and materials. Unlike other methods that use ozone, there is no exposure limit to the hydrogen water. Notably, all water and all aqueous solutions contain both H⁺ and OH⁻ ions. If the quantity of H⁺ exceeds that of the OH⁻, the water is said to be acidic. And, if there are more OH⁻ ions than H⁺, the water is alkaline. Pure water, which contains equal numbers of both ions, is said to be neutral. Whether water is acidic or alkaline, it will generally contain equal numbers of positive and negative electric charges. Since alkaline water contains an excess of OH⁻ ions, it must also contain some other kind of positive ion in addition to H⁺ in order to equalize the opposite charges. This extra positive ion is generally a metal ion such as sodium, calcium or magnesium. Similarly, acidic water generally contains negative ions in addition to OH⁻; the most common of these is bicarbonate HCO3⁻. This means that waters whose pH differs from 7 is not “pure” in the chemical sense. Alternatively, the system and method of the present invention can utilize other mechanisms for generating the hydrogenated water, or a quantity of hydrogenated water can be provided from an outside source and deposited into the system. For example, the hydrogen subsystem may generate hydrogen water by bubbling gaseous hydrogen through a contained volume of water, or may store a quantity of externally generated hydrogen water for delivery.

Electrolysis refers to the breakdown of a substance by passing an electric current through it. This is accomplished by placing a pair of oppositely charged electrodes (such as a platinum catalyst or some other un-reactive metal) in the water solution. Positive ions that find themselves close to the negative electrode will acquire electrons, and negative ions near the positive electrode will lose electrons. In the case of pure water, as depicted in FIG. 4, the reactions are as follows: At the negative electrode, 2 H⁺+2e⁻→H₂, but since the H⁺ ions come from water, the overall reaction is 2 H₂O+2e⁻→H₂+2 OH⁻. And at the positive electrode, 4 OH⁻→4e⁻+2 H₂O+O₂, but since the OH⁻ ions come from water, the overall reaction is 2 H₂O→4e⁻+O₂+4H⁺. In the case of the hydrogen water device, the generation of ions is safe as the H⁺ and the OH⁻ will recombine if there is nothing to act on (e.g., H⁺+OH⁻→H₂O). Generally, when tap water is used, chlorine may be present in the water. If this is the case, chloride ions at the anode are oxidized to elemental chlorine. If some of this chlorine is allowed to combine with some of the hydroxide ions produced at the cathode, it forms hypochlorous acid (HOCL), a weak acid and an oxidizing agent. In example embodiments, the hydroxyl radicals that are produced from the reaction provide for acting as a disinfectant.

In example applications of use, the humidification and treatment systems of the present invention may be utilized to clean and disinfect various equipment or materials, such as for example, display cases, refrigeration coils, and associated equipment used in connection with perishable items such as produce, milk, vegetables, etc. In alternate applications, the system 10 may be used to disinfect medical areas or doctor's offices, wine/grape containment areas, trucks used to deliver perishables, schools, florist shops, or other areas where it may be desirable to clean, disinfect, or provide hydration/humidification. In certain applications, the provision of moisture or humidification in the form of a mist or fog is advantageous for maintaining perishable items such as produce, flowers, cannabis, meat, seafood, other organic materials, or other perishable items, that have a desired water content. In alternate applications, the mist or fog is merely a delivery media for distribution of the disinfecting agent in the form of hydrogen-rich water. Without limitation to theory, the hydrogenated water is believed to kill pathogenic organisms (e.g., fungi, mold, mildew, bacteria, yeast, etc.) by lysis. Such pathogens may naturally occur or otherwise be present in or on produce or other food products, on or around equipment and containment areas (e.g., display cases), and/or in the air or environment associated therewith. By delivery of disinfecting hydrogen water via misting or fogging, the entire environment within, on or around a treated item or space can be disinfected and cleaned.

In addition to spray nozzles, various other dispersion or discharging mechanisms can be utilized to convert a coherent quantity of hydrogen water to a mist or fog of droplets or microdroplets of hydrogen water, such as for example, ultrasonic misters or foggers, steam generation, droplet fractioning, or other misting, fogging, spraying or other methods of dispersion.

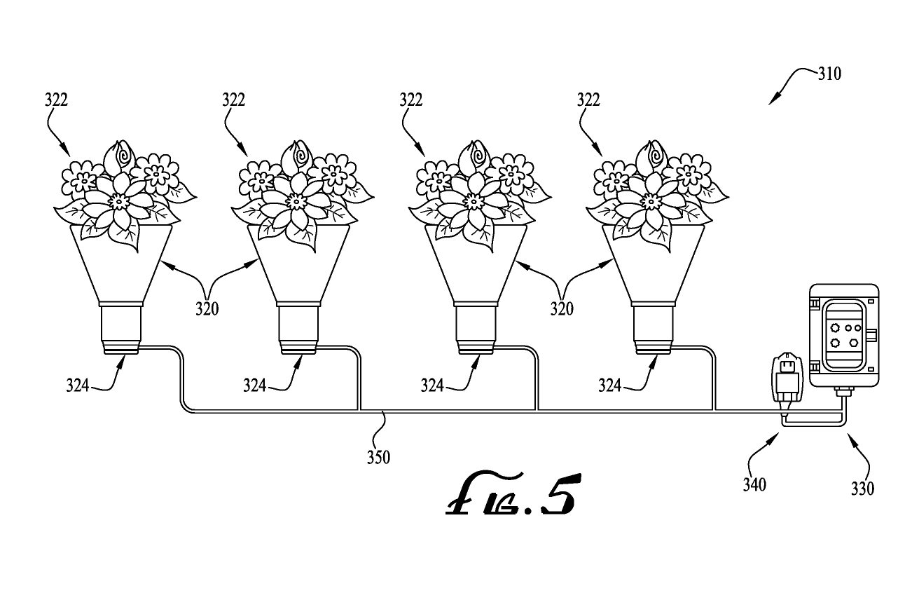

FIG. 5 shows a system 310 and method for preserving flowers, floral arrangements, produce and/or other plant matter or other perishable items using hydrogen-rich water. One or more, and preferably a plurality of containers such as floral vases 320 each contain a quantity of hydrogen rich water and flowers, floral arrangements and/or other plant matter 322 at least partially contained therein and having stem or other portions in contact with the hydrogen rich water. Each vase 320 optionally comprises a hydrogen water cell 324 for generating hydrogen to enrich the water contained therein. A power source and controller 330, 340 are connected to the hydrogen water cells 324 by wiring 350. In alternate embodiments, a portable hydrogen water cell is inserted into the vases and into contact with water contained therein to generate hydrogen rich water in each vase. In example forms, the hydrogen water cells 324 generate hydrogen water by electrolysis using charged electrodes, as described above. In still further alternate embodiments, the hydrogen rich water is circulated or otherwise delivered to the vases 320 from a shared source of hydrogen rich water, and piping or fluid delivery conduits provide distribution of the hydrogen rich water from the source to the vases or containers.

In an example method of use of the system 310 for preserving flowers, a quantity of water is placed in each of the vases 320. The hydrogen water cells 324 are operated, intermittently or continuously, to generate hydrogen and convert the water to hydrogen rich water. Flowers or other plants 322 are placed with their stems at least partly submerged in the hydrogen rich water within the vases 320. Alternatively, hydrogen rich water from a shared source is delivered to the vases, and/or continuously or periodically circulated through an array of vases via a hydrogen rich water delivery system. In example embodiments, the system 310 can deliver hydrogen water to cut stems or stalks of flowers or other cut plant material, or alternatively can deliver hydrogen water to the roots of live growing plants for example in hydroponic growth systems wherein the containers 320 comprise grow trays or root containers.

While the invention has been described with reference to preferred and example embodiments, it will be understood by those skilled in the art that a variety of modifications, additions and deletions are within the scope of the invention, as defined by the following claims.

What is claimed is:

1. A treatment system for controlling pathogens, the system comprising a hydrogenated water subsystem delivering a quantity of hydrogenated water from a source, a delivery subsystem, and a dispersing subsystem for converting the hydrogenated water to a mist or fog and delivery of that mist or fog of hydrogenated water to a treated object.

2. The treatment system of claim 1, wherein the hydrogenated water subsystem comprises an electrolysis device for converting a supply of regular water into hydrogenated sterilized water.

3. The treatment system of claim 1, wherein the dispersing subsystem comprises at least one misting or fogging nozzle.

4. The treatment system of claim 1, further comprising a control subsystem for controlling one or more parameters of the delivery of the mist or fog of hydrogenated water to the treated object.

5. The treatment system of claim 1, wherein the treated object is a perishable selected from produce, meat, seafood, flowers and perishable items having a desired water content.

6. A humidification system comprising a regular water supply, an electrolysis device for converting regular water from the regular water supply into hydrogenated water, and at least one nozzle for discharging the hydrogenated water in the form of a mist or fog.

7. The humidification system of claim 6, further comprising a control system for controlling at least one parameter of the mist or fog delivery.

8. The humidification system of claim 6, further comprising a misting track comprising a plurality of nozzles for discharging the hydrogenated water in the form of a mist or fog.

9. A method of preserving and/or disinfecting a treated article or area, the method comprising providing a quantity of hydrogenated water, converting the hydrogenated water to a mist or fog, and delivering the mist or fog of hydrogenated water to the treated article or area.

10. The method of claim 9, wherein the step of providing a quantity of hydrogenated water comprises converting regular water into hydrogenated water using an electrolysis device.

11. The method of claim 9, wherein the treated article comprises perishables.

12. The method of claim 9, wherein the step of converting the hydrogenated water to a mist or fog comprises discharging a stream of the hydrogenated water through a misting or fogging nozzle.

13. A system for preserving a perishable item, the system comprising at least one container for containing a quantity of hydrogen rich water and the perishable item with at least a portion of the perishable item retained in contact with the hydrogen rich water.

14. The system of claim 13, wherein each of the containers comprises a hydrogen water cell for generating hydrogen for the hydrogen rich water.

15. The system of claim 13, wherein the hydrogen rich water is delivered to the plurality of containers from a shared source of hydrogen rich water, the system including the shared source of hydrogen rich water, a plurality of containers, and fluid delivery conduits for distribution of the hydrogen rich water from the source to the containers.

16. The system of claim 13, wherein the containers comprise hydrogen rich water generating vases.

17. The system of claim 13, wherein the perishable item comprises a cut flower.

18. A method for preserving perishable items, the method comprising placement of the perishable items in at least one container with at least a portion of the perishable items in contact with a quantity of hydrogen rich water contained in the container.

19. The method of claim 18, further comprising generating the hydrogen rich water in the container.

20. The method of claim 18, wherein the hydrogen rich water is generated by electrolysis in the container.
